(19) United States
(12) Patent Application Publication
(10) Pub. No.: US 2009/0297481 A1
(43) Pub. Date: Dec. 3, 2009

(54) ANIMAL FEED SUPPLEMENTS AND USES THEREOF

(76) Inventor: Owen Jay Powlen, Kewanna, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/406,639

(22) Filed: Mar. 18, 2009

Related U.S. Application Data
(63) Continuation of application No. PCT/US2007/078860, filed on Sep. 19, 2007.
(60) Provisional application No. 60/845,722, filed on Sep. 19, 2006.
(60) Provisional application No. 60/845,857, filed on Sep. 20, 2006.

(51) Int. Cl. A61K 35/74

(52) U.S. Cl. 424/93.3

(57) ABSTRACT

Described feed supplement compositions containing beneficial viable microorganisms and potentially other beneficial nonviable materials, and methods of use thereof.

### REFERENCE TO RELATED APPLICATIONS

The present application claims the benefit of U.S. Provisional Patent Application Ser. No. 60/845,722, Filed Sep. 19, 2006 and U.S. Provisional Patent Application Ser. No. 60/845,857, Filed Sep. 20, 2006 both entitled ANIMAL FEED SUPPLEMENTS AND USES THEREOF which is hereby incorporated by reference in its entirety.

### BACKGROUND

The present invention relates generally to animal feed supplements, and in particular embodiments to animal feed supplements that include microbial or microbial-derived materials.

As further background, the breeding and maintenance of domestic animals such as beef cattle, dairy cows, pigs, fowls and others, has developed as a highly sophisticated and competitive industry. In connection with these industries, it is known to feed the animals so-called “direct fed microbial” (DFM) products in order beneficially affect the breeding or maintenance program. Oftentimes, the DFM composition will contain viable organisms such as bacteria, or preparations from fermentations of such organisms containing enzymes and potentially other beneficial substances.

Despite advancements in the area, needs exist for improved and alternative DFM compositions and methods for feeding to animals, including humans. Desirably, these compositions and methods would provide enhanced performance in one and preferably several aspects of animal breeding and/or maintenance, including for example one or more of feed utilization, milk production, animal health, fiber digestion, and potentially others. The present invention is addressed to these needs.

### SUMMARY

Accordingly, in one aspect, the present invention provides a method for enhancing milk production from a herd of dairy cows. The method includes administering to dairy cows in the herd a daily feed supplement comprising viable Lactobacillus bacteria, viable Streptococcus faecium bacteria, and L-phase Lactobacillus bacteria. In certain embodiments, at least 10⁸ viable Lactobacillus bacteria and 10⁸ viable Streptococcus faecium bacteria are fed to the cows individually, each day. In additional embodiments, the daily feed supplement can also comprise viable yeast and/or a yeast fermentation product, and/or viable photosynthetic bacteria or a photosynthetic bacteria fermentation product. In still further embodiments, the feed supplement composition comprises microbial sugars and/or mannanoligosaccharides. The daily feed supplement can be effective to increase the butterfat weight content of milk produced by the herd, e.g. by at least about 1%, and/or to increase the volume of milk produced per day by the herd, e.g. by at least about 1%.

In another embodiment, provided is a method for maintaining a group of domesticated animals. The method includes administering to animals in the group a daily feed supplement that comprises viable Lactobacillus bacteria, viable Streptococcus faecium bacteria, and L-phase Lactobacillus bacteria. The animals can for example be porcine, bovine or poultry. The feed supplement composition can further include viable yeast or a nonviable yeast fermentation product, and/or viable photosynthetic bacteria or a nonviable fermentation product thereof. As well, the feed supplement composition can include microbial sugars.

In another embodiment, the invention provides a method for maintaining a group of domesticated animals. The method includes administering to individual animals in the group a daily feed supplement comprising both viable Lactobacillus bacteria and L-phase Lactobacillus bacteria.

In a further embodiment, the invention provides a method for maintaining a group of domesticated animals that includes administering to individual animals in the group a daily feed supplement comprising at least 1×10⁹ viable Lactobacillus bacteria and at least 1×10⁹ viable Streptococcus faecium bacteria.

Another embodiment of the invention provides a method for maintaining a group of domesticated animals that includes administering to animals in the group a daily feed supplement comprising viable Streptococcus faecium bacteria and L-phase Lactobacillus bacteria.

In a further embodiment, the invention provides a feed supplement composition for domesticated animals. The composition comprises at least 1×10⁹ viable Lactobacillus bacteria per gram and at least 1×10⁹ viable Streptococcus faecium bacteria per gram. The composition can also include L-phase Lactobacillus bacteria, viable yeast or a yeast fermentation product, viable photosynthetic bacteria or a fermentation product thereof, or combinations of any of these. The composition can further comprise microbial sugars and/or mannanoligosaccharides.

In a further embodiment, the invention provides a feed supplement composition for domesticated animals, wherein the composition includes at least viable Lactobacillus bacteria and L-phase Lactobacillus bacteria. The composition can also include viable yeast, a nonviable yeast fermentation product, viable photosynthetic bacteria, a nonviable photosynthetic bacteria fermentation product, or any combination of these materials. The composition can also include microbial sugars.

In a still further embodiment, the present invention provides a feed supplement composition for domesticated animals. The composition includes viable Streptococcus faecium bacteria and L-phase Lactobacillus bacteria. This composition can also include viable Lactobacillus bacteria, viable yeast, a nonviable yeast fermentation product, viable photosynthetic bacteria, a nonviable photosynthetic bacteria fermentation product, microbial sugars, or any combination of these.

In a further embodiment, the invention provides a feed supplement composition. The composition comprises at least 1×10⁸ viable Lactobacillus bacteria per gram; and at least one of: (i) 1×10⁸ viable Streptococcus faecium bacteria per gram; and (ii) 1×10⁸ viable Bifidobacterium bacteria per gram, desirably Bifidobacterium bifidus. In a preferred form, the composition includes both (i) and (ii) above. In a further preferred form, the composition includes both (i) and (ii), and the Lactobacillus bacteria include both Lactobacillus acidophilus and Lactobacillus casei, each preferably present at a level of at least 10⁸ viable bacteria per gram, and more preferably at a level of at least 10⁹ viable bacteria per gram. The composition can also include L-phase Lactobacillus bacteria, viable yeast and/or a yeast fermentation product, viable photosynthetic bacteria or a fermentation product thereof, or combinations of any of these. The composition can further comprise microbial sugars and/or mannanoligosaccharides and/or one or more additional components as described herein.

Additional embodiments of the invention pertain to feed compositions that include feed ration materials for domesticated animals (e.g. plant-derived feeds such as corn, hay, etc. and silages thereof) in combination with inventive feed supplements described herein.

Still further embodiments of the present invention relate to articles of manufacture that comprise a feed supplement composition of the invention as described herein contained within packaging that preserves the viability of microorganisms present in the composition. Related methods of use of such articles of manufacture in methods to provide feed supplementation to animals also form a part of the present invention.

Additional embodiments of the present invention as well as features and advantages thereof will be apparent from the descriptions herein.

### DETAILED DESCRIPTION

For the purposes of promoting an understanding of the principles of the invention, reference will now be made to certain embodiments thereof and specific language will be used to describe the same. It will nevertheless be understood that no limitation of the scope of the invention is thereby intended, such alterations and further modifications in the embodiments described, and such further applications of the principles of the invention as described herein being contemplated as would normally occur to one skilled in the art to which the invention relates.

As disclosed above, in certain of its aspects, the present invention provides DFM compositions, articles of manufacture and methods relating to animal feed supplements for domesticated animals or humans. Compositions, articles and methods of the invention involve the use of viable, beneficial bacterial cells. These bacterial cells can include Lactobacillus bacteria and/or Streptococcus faecium bacteria. In certain embodiments, L-form Lactobacillus bacteria are included in the composition. Additional beneficial ingredients can also be included, for example including microbial sugars, yeast or yeast components (e.g. extracts), photosynthetic bacteria or components thereof, and/or mannanoligosaccharides. As well, other bacteria may be included, for example including Bifidobacterium bacteria such as Bifidobacterium bifidus.

Viable Lactobacillus bacteria cells that can be used in DFM compositions of the invention include, for example, Lactobacillus acidophilus, Lactobacillus bulgaricus, Lactobacillus casei, Lactobacillus cellobiosus, Lactobacillus confusus, Lactobacillus fermentum, Lactobacillus lactis, Lactobacillus leichmannii, Lactobacillus ruminis, Lactobacillus bifidus, and Lactobacillus thermophilus. Lactobacillus acidophilus is a preferred bacterium for use in compositions of the invention, alone or combined with one, two or three additional types of Lactobacillus bacteria from the above list. In certain preferred embodiments, Lactobacillus acidophilus is used in combination with Lactobacillus casei, e.g. in approximately equal amounts.

In certain embodiments, DFM compositions and methods of the invention involve the use of Streptococcus faecium bacteria. These bacteria are known to have special affinity for fibrous materials such as lignins, making these materials a primary food source for the bacteria. In this regard, Lactobacillus casei is also a fiber-digesting organism, and can be used in the place of or in addition to Streptococcus faecium in certain inventive variants herein, e.g. in combination with one or more additional Lactobacillus bacteria, e.g. from among those listed above. Other bacteria that utilize fibers such as lignins as their primary food source can also be used.

In certain embodiments, compositions of the invention also include L-phase Lactobacillus bacteria. These bacteria can be chosen from any of those Lactobacillus species listed above. Again, Lactobacillus acidophilus is one preferred bacterium that can be used.

Compositions of the invention can also include microbial sugars. These can include one or more of, and preferably all of, D-Erythrose, DL-Arabinose, D(−) Ribose, D-Lyxose, a-D-Galactose pentarcethte, D(+) Mannose, D-Gulse, D-Idose, aD-Talose, D-Altrose, B-D-Allose, D-(+)-Sorbose, D-Tagatose, Maltose, a-D(+) Melibiose, D-(+)Cellobiose, and D-(+) Trehalose.

As well, in certain embodiments, compositions of and used in the invention include viable yeasts or nonviable components (e.g. enzymes) derived therefrom. Examples of such yeasts include members of the genus Saccharomyces, such as Saccharomyces cerevisiae, Saccharomyces uvarum, Saccharomyces faecalis, Saccharomyces fragilis, Saccharomyces lactis, etc.; members of the genus Candida such as Candida utilis, Candida tropicalis, Candida rugosa, Candida perculirosa, etc.; and the like. In addition to viable forms, nonviable products obtained by fermentation of materials with yeast, followed by killing the viable yeasts and potentially extracting desirable components, can be used in compositions of the present invention. When used, viable yeast organisms can be included, for example, at a level of at least about 10⁸ colony forming units per gram (cfu/gm), for example in the range of about 10⁸ to about 10¹² cfu/gm. Enzymes incorporated in the composition can be derived from these or other yeast, or other organisms or recombinant sources, and can include for example cellulase, hemicellulase, amylase, protease, lipase, xylanase, esterase, or combinations including some or all of these enzymes.

Feed supplement compositions of the invention can also include viable photosynthetic bacteria, and/or nonviable products that can be obtained therefrom. Illustrative photosynthetic bacteria that can be used include, for example, members of the family Rhodospirillaceae such as Rhodospirillum rubrum, Rhodospirillum tenue, Rhodospirillum fulvum, Rhodospirillum molischianum, Rhodospirillum photometricum, Rhodopseudomonas palustris, Rhodopseudomonas gelatinosa, Rhodopseudomonas capsulata, Rhodopseudomonas viridis, Rhodopseudomonas acidophila, Rhodopseudomonas sphaeroides, Rhodomicrobium vannielii, etc.; members of the family Chromatiaceae such as Chromatium vinosum, Chromatium okenii, Chromatium warmingii, Chromatium bunderi, Chromatium minus, Chromatium violascens, Chromatium weissei, Chromatium gracile, Thiocystis galatinosa, Thiocystis violacea, Thiospirillum sanguineum, Thiospirillum jenense, Thiospirillum rosenbergii, etc.; and the like. Among others, so-called purple non-sulfur bacteria belonging to the genera Rhodospirillum and Rhodopseudomonas can be used. Moreover, two or more species or strains of these bacteria are used in certain embodiments of the invention.

In addition, mannonoligosaccharides may be incorporated in compositions and/or feeding regimens of the present invention. These may be derived from the cell walls of yeast, such as those mentioned above. Although not intending to be bound by any theory, it is thought that mannonoligosaccharides can bind to the fimbria of pathogenic bacteria and can favor competitive exclusion by beneficial microorganisms.

Also, compositions of the invention can include amounts of viable bacteria traditionally known to aid in the fixation of nitrogen, or nonviable fermentation products thereof. These may include, for instance, one or more of Azotobacter vinelandii, Bacillus megaterum, Rhizobium leguminosarm or the like. Trichoderma virde or nonviable fermentation products thereof can also be included to facilitate fiber decomposition.

Compositions of the invention can also include a suitable carrier material, desirably a dry, powdered carrier. Preferably, the carrier includes a dry, powdered carbohydrate source such as whey, e.g. sweet whey. The carrier can also include lactose, e.g. as a component of the whey and/or added separately. Further, the composition can include one or more other sugars such as dextrose. The carrier can also include a drying agent, in powder form, such as Sipernet, a material that is commercially available from Phar-More Biochemical. Use of such a drying agent is particularly advantageous where the composition is initially formulated using bacterial compositions in liquid form. Furthermore, the carrier should be free from agents that would detrimentally interfere with the viability of the bacteria in the composition.

In additional embodiments, the carrier can be a liquid material, desirably a liquid hydrocarbon such as an oil, that preserves the viability and/or other activities of the ingredients of the composition. Illustrative oils that may be used include for example vegetable oil(s) such as corn oil and/or soybean oil. When a liquid carrier is used, it will preferably be free or essentially free from water. When compositions of the invention are in liquid form, they can be used in inventive feeding and feed preparation methods, in which the liquid composition is pumped and potentially metered into a feed ration material which is mixed and provided to animals for consumption.

In certain embodiments, compositions of the invention containing two (2) or more of, and potentially all of, the above-noted ingredients can be prepared, for example, by dry blending the separate ingredients. Alternatively, some or all of the materials can be present in, or incorporated in, a fermentation product of one, two, or more microorganisms. Such a fermentation product can then be processed to dry form so as to maintain viability of the microorganisms, or so as to kill the microorganisms and nonetheless provide a composition containing enzymes and/or other beneficial components. In this regard, techniques for culturing these microorganisms and processing the same to viable or nonviable compositions are known and their use within the present invention will be within the purview of those of ordinary skill in the art.

In certain embodiments, compositions of the invention will contain at least one type of viable Lactobacillus bacterium and at least one type of Streptococcus faecium bacterium at a relatively high concentration. It is contemplated that compositions containing both Lactobacillus bacteria and Streptococcus faecium bacteria at a level of at least 10⁶ viable bacteria per gram will be beneficial in certain instances. In selected embodiments, each of these bacterial types will be present at a level of at least 10⁸ viable bacteria per gram, and in certain additional embodiments at least 10⁹ viable bacteria per gram. In most embodiments, it is contemplated that the level of each of these bacterial types will be in the range of 10⁸ to about 10¹⁴ viable bacteria per gram, more typically about 10⁸ to about 10¹² viable bacteria per gram. In certain embodiments, the animal feed composition comprises at least about 5×10⁹ viable Lactobacillus bacteria per gram and at least about 3×10⁹ viable Streptococcus faecium bacteria per gram As to ratios of viable bacteria, when both viable Lactobacillus and viable Streptococcus faecium or other fiber-digesting bacteria are included, their ratio will generally be in the range of 2:8 to 8:2, respectively, more typically in the range of about 6:4 to about 4:6, respectively. In certain preferred embodiments, this respective ratio will be about 6:4 to about 5:5. Similarly, when a viable Bifidobacterium such as Bifidobacterium bifidus is present, the viable Lactobacillus and viable Bifidobacterium can be present in a ratio of 2:8 to 8:2, respectively, more typically in the range of about 6:4 to about 4:6, respectively. In certain preferred embodiments, this ratio of viable Lactobacillus and viable Bifidobacterium will be about 6:4 to about 5:5. Still further, when the viable Lactobacillus bacteria include both viable Lactobacillus acidophilus and viable Lactobacillus casei, the viable Lactobacillus acidophilus and viable Lactobacillus casei can be present in a ratio of 2:8 to 8:2, respectively, more typically in the range of about 6:4 to about 4:6, respectively. In certain preferred embodiments, this ratio of viable Lactobacillus acidophilus to viable Lactobacillus casei will be about 6:4 to about 5:5. It will be understood however that other ratios than those specified above can be used within broader aspects of the invention. In addition, in certain embodiments, the viable bacteria in the composition can be encapsulated in order to provide increased protection and preservation of their viability.

As noted above, in certain forms, compositions of the invention will include an amount of L-form or L-phase Lactobacillus bacteria. L-forms are bacterial spheroplasts or protoplasts originating from normal bacteria following partial (spheroplasts) or complete (protoplasts) removal of the cell wall. Methods for preparing such L-form Lactobacillus bacteria are known in the art. These methods generally involve the culture of the viable bacteria, followed by partial or complete removal of the cell walls from the bacteria by acid hydrolyses, antibiotics, salt, serum, or other suitable means. The result is a transformation of the bacteria to the L-form. When L-form fermentation products are fed to domestic animals, beneficial changes to the intestinal tract can occur, including, for example, via reversion of the L-form to viable cellular form Lactobacillus bacteria. In certain embodiments of the invention, an L-form Lactobacillus fermentation product is incorporated in the composition at a level of at least about 0.1 gram for every 100 grams (0.1% by weight) of the overall composition, e.g. in the range of about 0.1 to about 5 grams for every 100 grams of the overall composition (0.1% to 5% by weight). In selected embodiments, the L-form Lactobacillus fermentation product will be included at a level of about 0.4 grams for every 100 grams of the overall composition. It will be understood, however, that other levels may be used.

Compositions of the invention can be used as feed supplements for a wide variety of animals. These include, for example, bovine animals, such as beef cattle, dairy cows, and others, porcine animals, such as hogs raised for meat production, ovine animals, such as sheep, poultry, such as chickens, ducks or turkeys raised for meat production, caprine animals, such as goats, aquatic animals such as fish, crayfish or crustaceans, and others, including companion animals such as dogs. The animals can, for instance, be adults, adolescents or infants. The administration of compositions of the invention to these animals can be achieved in a variety of ways. The substances to be administered can be administered separately in effective amounts, or some or all of the substances can be administered in a combined composition. Dry mixtures will be suitable for these purposes.

In certain embodiments, the diet of the animals being treated will be supplemented each day with the DFM materials identified herein. This can be accomplished, for example, by providing the animal with feed rations containing the DFM materials. Such feed rations, illustratively plant-derived rations such as corn, hay, or silages thereof, also form a part of the present invention. This DFM-supplemented feed can be provided once, twice, or more times per day. A single DFM-supplemented feeding per day is preferred, e.g. in the morning. In one preferred mode, feed of the animals is supplemented with a dry mixture or coating of the DFM composition(s) to provide the beneficial substances in the desired amounts. Alternatively, the DFM composition(s) can be fed in a bolus, capsule, or other unit dose form. When feeding the supplement composition in the animals' daily rations, the composition or compositions can be blended with the feed rations in conventional hoppers or mixers from which the feed is dispensed. In this regard, a daily supply of feed can be blended with compositions of the invention, or sufficient blended feedstuff containing compositions of the invention can be prepared to feed the animals over a number of days or weeks. In certain embodiments, the supplemented feed will be controlled in production and volume so as to be fed to the animals within about three days after blending with the DFM material(s). These and other variations in the feeding or other administration aspects will be apparent to skilled artisans. Additionally, it will be understood that animal feed rations often contain toxins, for example Aflatox and/or vomitoxin, that detrimentally impact animal health, productivity and other factors. Supplementation with DFM material(s) in accordance with the present invention provides substantial benefits as described herein to animals that consume toxin-containing feed rations.

In certain modes of operation, the DFM supplement regimen described herein can be undertaken in combination with other treatments to enhance breeding or maintenance of the animals. For instance, DFM supplementation as described herein can be undertaken in combination with a hormonal treatment, e.g. of dairy cows to enhance milk production. In one embodiment, DFM supplementation as described herein is used in combination with bovine somatotropin (potentially recombinant) administration in dairy cows to further enhance production by increasing milk volume and/or butterfat content.

DFM composition(s) of the invention, in desirable forms, are packaged in containers that preserve the viability of the viable organisms therein. These can include for example sealed film or foil packages containing the materials in dry, stable form. These packages can be sized such that their contents will be used in a short period of time after opening, e.g. on the same day or within about a week of opening. In this manner, viability of the organisms when fed or otherwise administered to the animals can be facilitated. These packages can bear labels identifying the contents as feed supplements, providing a listing of ingredients contained within the package, providing a recommended amount and frequency of feeding the contents to an animal (e.g. specifying one of those types of animals identified herein), and/or providing an expiration date for the package, and the like. Such packaged compositions are also considered to be articles of manufacture constituting aspects of the present invention.

DFM supplementation as described herein can provide one or several benefits to the animal(s), including humans or domesticated animals such as beef cattle, dairy cows, swine, sheep, goats, equines such as horses, fowl, show animals, and others. For instance, this DFM supplementation can improve the overall health of the animals (e.g. as evidenced by decreased death loss, improvements in breeding efficiency, improvements in body conditioning scores, etc.), can increase the efficiency of feed utilization (e.g. in the generation of milk or meat in bovines, porcines, fowls or other animals), can increase fiber digestion (e.g. as evidenced by lower fiber content in solid wastes from the animals), and/or can decrease the volume of solid wastes from the animals thereby allowing for increased herd size for a given facility. In certain inventive aspects, substantial enhancements in milk production from dairy cows, individually or as a herd, can be achieved using DFM feed supplement compositions of the invention. Illustratively, milk volume production and/or butterfat contents of milk can be improved, e.g. by at least about 1% or more, and in certain embodiments by at least about 2% or more, e.g. usually in the range of about 1% to about 10%. The above-mentioned improvements in animals can also be exhibited when the animals are under stress, including for example caused by feed toxins, illness, side-effects of other therapeutic regimens, environmental conditions such as excessive heat, and others.

For the purpose of promoting a further understanding of certain aspects of the invention, the following Examples are provided. It will be understood that these Examples are illustrative, and not limiting, of aspects of the invention.

### EXAMPLE 1

A herd of dairy cows (Holstein, between 667 and 688 cows milked per day) was cycled weekly on and off a feed supplement composition of the invention. The feed supplement composition was added as a dry composition to the feed rations (including corn and corn silage)) for the herd. The feed supplement composition was a mixture having a sweet whey/lactose carrier containing approximately 5×10⁹ cfu of viable Lactobacillus acidophilus per gram and approximately 3×10⁹ cfu of viable Streptococcus faecium per gram, L-form Lactobacillus (˜0.4 grams of L-form fermentation product per 100 grams of overall composition), a thermophilic fungi fermentation product, and the following microbial sugars: D-Erythrose, DL-Arabinose, D(−) Ribose, D-Lyxose, a-D-Galactose pentacetate, D(+) Mannose, D-Gulose, D-Idose, aD-Talose, D-Altrose, B-D-Allose, D-(+)-Sorbose, D-Tagatose, Maltose, a-D(+) Melibiose, D-(+)Cellobiose, and D-(+) Trehalose.

The feed supplement composition was fed at a rate of one (1) gram per day per cow. The results are tabulated in Table 1, attached

As can be seen from Table 1, the absolute data trending reflects that the feed supplement composition and method of the invention are effective in enhancing milk production by the dairy cows. An increase in milk production was evident immediately after introducing the inventive feed supplement composition to the herd diet, and that increase held during the off week that followed. In week four the temperatures increased dramatically, leading to a drop in milk volume production; however, butterfat content increased and held up 13 points.

### EXAMPLE 2

A herd of dairy cows (Holstein, between 954 and 1017 cows milked per day) was cycled weekly on and off a feed supplement composition of the invention, using a procedure as describe in Example 1 above. The results are tabulated in Table 2, attached.

As can be seen from Table 2, the absolute data trending in Table 2 reflects that the feed supplement composition and method of the invention are effective in enhancing milk production by the dairy cows. Milk volume production increased during the weeks on the inventive feed supplement and receded during the off week. Butterfat production also rose during the first week on the inventive feed supplement and even more dramatically during the second week on the feed supplement. These production increases were achieved despite daytime temperatures rising near or above 100 degrees F.

### EXAMPLE 3

A herd of dairy cows (Holstein, between 667 and 694 cows milked per day) was cycled weekly on and off a daily feed supplement composition of the invention, using a procedure as describe in Example 1 above. The results are tabulated in Table 3, attached.

As can be seen in the data in Table 3, results of this experimental trial were somewhat inconsistent. Milk production increased in the first week on the inventive feed supplement, and then receded in the following off and on weeks.

### EXAMPLE 4

600 head of dairy cows were put on a daily feed supplement including the inventive feed supplement as described in Example 1, in addition to Bio Cycle Plus (½ ounce per head per day) and BioFresh Bolus (Marketed by Select Sires, Inc., manufactured by Agrarian Marketing Company, Inc.). Bio Cycle Plus includes yeast culture, calcium carbonate, dried egg product, aspartic acid, lactic acid, calcium lactate, malic acid, acetic acid, thiamine mononitrate, citric acid, calcium pantothenate, fumaric acid, papain, saccharomyces cerevisiae (active dry yeast), dried Aspergillus oryzae fermentation extract, dried Aspergillus niger fermentation extract, sodium potassium tartrate, potassium iodide, silicon dioxide and sodium silico aluminate. It has the following guaranteed analysis (per ½ ounce dose):
Amylase (A. oryzae): 3750 ug of starch hydrolyzed/minute/gram
Protease (A. oryzae): 500 ug of protein hydrolyzed/minute/gram
Cellulase (A. niger): 200 ug of cellulose broken-down/minute/gram
Lipase (A. oryzae): 150 ug of triglycerides hydrolyzed/minute/gram
Thiamine: 400 mg/pound
Citric Acid: 400 mg/pound
   BioFresh Bolus includes yeast culture, Saccharomyces cerevisiae (active dry yeast), potassium iodide, dried Aspergillus oryzae fermentation extract, dried Aspergillus niger fermentation extract, dried egg product, lactic acid, calcium lactate, malic acid, acetic acid, tartaric acid, fumaric acid, dicalcium phosphate, ferrous sulfate, magnesium sulfate, zinc proteinate, copper proteinate, manganese proteinate, cobalt glucoheptonate, choline chloride, sodium selenite, silicon dioxide, vitamin A acetate, d-activated animal sterol (source of vitamin D-3), ascorbic acid, niacinamide, calcium iodate, biotin, calcium pantothenate, menadione dimethylpyrimidinol bisulphate (source of vitamin K3), vitamin B12 supplement, riboflavin, thiamine mononitrate, folic acid, cobalt sulfate, pyridoxine, mineral oil, potassium citrate, ethyl butyrate, vanilla and milk flavor, artificial sweetener. It has a guaranteed analysis (per bolus) of:
Amylase (A.oryzae): 30,000 ug of starch hydrolyzed/minute/gm
Protease (A.oryzae): 4,000 ug of protein hydrolyzed/minute/gm
Cellulase (A.niger): 1,600 ug of cellulose broken-down/minute/gm
Lipase (A.oryzae): 1,200 ug of triglycerides hydrolyzed/minute/gm
Pectinase (A.niger): 800 ug of pectin hydrolyzed/minute/gm
The feed for the cows was highly toxic (Aflatox 15 ppb; vomitoxin 527 ppb; T-2 34 ppb). At the beginning of the trial, the average milk production of the cows was 74 lbs./head/day. The average death loss in the three months prior to the trial was 20 head/month. About two weeks into the trial, an additional 300 head were added to the trial. The average milk production six months into the trial was 80 lbs/head/day.

### EXAMPLE 5

A dairy cow herd of 108 head (Holsteins) was put on a daily feed supplement as described in Example 1. The average milk production for the herd going into the trial was 80 lbs/head/day. After nine days on the feed supplement (2 grams/head/day, fed once daily with rations), the average milk production was 85.5 lbs/head/day. After three days off the feed supplement, the herd was returned to the supplement at a rate of 1 gram/head/day for four days. The average milk production for the herd going into this second on-supplement stage of the trial was 82 lbs/head/day. The average milk production at the end of the four on-supplement days was 86 lbs/head/day.

### EXAMPLE 6

A dairy cow herd of 160 head (Holsteins) was put on a daily feed supplement as described in Example 1 at a rate of 1 gram/head/day. Leading up to and during the trial, the herd was also on a standard 14-day interval regimen of recombinant bovine somatotropin (rBST). One day before the bottom of the rBST interval, the herd was put on the daily feed supplement as described in Example 1 above (fed once daily with rations). The average milk production for the herd going into the feed supplement trial was 68.75 lbs/head/day, with an average butterfat content of 3.95%. After three days on the feed supplement (1 gram/head/day), the average milk production was 71.78 lbs/head/day, with an average butterfat content of 4.16%. The feeding of the supplement was then suspended. One day later, the average butterfat content had dropped to 4.01%. After 18 days off the feed supplement, the herd was put back on the supplement for an additional 15 days. The average milk production going into the second on-supplement phase was 68.74 lbs/head/day. At the end of this second phase, the average milk production was 70.5 lbs/head/day.

### EXAMPLE 7

Six dairy cows were subjected to testing to determine the impact of feeding a feed supplement as described in Example 1 upon fiber digestion in the cows. Prior to administering the feed supplement, fecal samples were collected from each cow via the rectum. After subsequent administration of the feed supplement described in Example 1 orally by bolus for 2 days, another fecal sample was obtained from each cow. Wet samples were weighed and then sieved through a 60 mesh screen by spraying water through the sample. Residue solids remaining on the screen was then collected, dried and weighed. The results reflected, on average, over a 20% reduction in solids fiber content of the samples. Additionally, feed residue analyses on the same manure samples showed increases in ash, calcium and magnesium, all indicators of increased fiber digestion.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

1. A method for enhancing milk production from a diary cow, comprising:
administering to a dairy cow a daily feed supplement comprising:
at least 10⁸ viable Lactobacillus bacteria;
at least 10⁸ viable Streptococcus faecium bacteria; and
L-phase Lactobacillus bacteria;
wherein said administering is effective to increase the volume of milk produced by the dairy cow per day, and to increase the level of fat in the milk.

2. (canceled)

3. The method of claim 1, wherein the viable Lactobacillus bacteria are Lactobacillus acidophilus bacteria.

4. The method of claim 3, wherein the L-phase Lactobacillus bacteria are Lactobacillus acidophilus bacteria.

5-7. (canceled)

8. The method of claim 1, wherein said daily feed supplement also comprises viable yeast, or a nonviable yeast fermentation product, or both.

9. The method of claim 1, wherein said daily feed supplement also comprises viable photosynthetic bacteria, or a nonviable photosynthetic bacteria fermentation product.

10. The method of claim 1, wherein said daily feed supplement also comprises microbial sugars.

11. The method of claim 10, wherein said microbial sugars contain one or more members selected from the group consisting of D-Erythrose, DL-Arabinose, D(−) Ribose, D-Lyxose, a-D-Galactose pentacetate, D(+) Mannose, D-Gulose, D-Idose, aD-Talose, D-Altrose, B-D-Allose, D-(+)-Sorbose, D-Tagatose, Maltose, a-D(+) Melibiose, D-(+)Cellobiose, and D-(+) Trehalose.

12-13. (canceled)

14. The method of claim 1, wherein said administering is effective to increase the volume of milk produced per day by the dairy cow by at least 1%.

15. The method of claim 1, wherein said administering is effective to increase the butterfat weight content of milk produced by the diary cow by at least 1%.

16. The method of claim 14 wherein said administering is effective to increase the butterfat weight content of milk produced by the diary cow by at least 1%.

17. A method for enhancing milk production from herd of diary cows, comprising:
administering to dairy cows in the herd a daily feed supplement comprising:
viable Lactobacillus bacteria;
viable Streptococcus faecium bacteria; and
L-phase Lactobacillus bacteria;
wherein said administering is effective to increase the average volume of milk produced per cow per day in the herd, and to increase the average level of butterfat in the milk produced by the cows in the herd.

18-32. (canceled)

33. A method for maintaining a herd of domesticated animals, comprising:
administering to animals in the herd a daily feed supplement comprising:
viable Lactobacillus bacteria;
viable Streptococcus faecium bacteria; and
L-phase Lactobacillus bacteria.

34. (canceled)

35. The method of claim 33, wherein the viable Lactobacillus bacteria are Lactobacillus acidophilus bacteria.

36. The method of claim 35, wherein the L-phase Lactobacillus bacteria are Lactobacillus acidophilus bacteria.

37-39. (canceled)

40. The method of claim 33, wherein said daily feed supplement also comprises viable yeast, or a nonviable yeast fermentation product, or both.

41. The method of claim 33, wherein said daily feed supplement also comprises viable photosynthetic bacteria, or a nonviable photosynthetic bacteria fermentation product, or both.

42. The method of claim 33, wherein said daily feed supplement also comprises microbial sugars.

43. The method of claim 42, wherein said microbial sugars contain one or more members selected from the group consisting of D-Erythrose, DL-Arabinose, D(−) Ribose, D-Lyxose, a-D-Galactose pentacetate, D(+) Mannose, D-Gulose, D-Idose, aD-Talose, D-Altrose, B-D-Allose, D-(+)-Sorbose, D-Tagatose, Maltose, a-D(+) Melibiose, D-(+)Cellobiose, and D-(+) Trehalose.

44-45. (canceled)

46. The method of claim 33, wherein said animals are bovine animals.

47. The method of claim 46, wherein said animals are beef cattle.

48. The method of claim 33, wherein said animals are porcine animals.

49. The method of claim 33, wherein said animals are fowl.

50-64. (canceled)

65. A method for maintaining a group of domesticated animals, comprising:
administering to individual animals in the group a daily feed supplement comprising at least 10⁸ viable Lactobacillus bacteria in combination with one or both of viable Streptococcus faecium bacteria and L-phase Lactobacillus bacteria.

66-67. (canceled)

68. A composition useful as a feed supplement for domesticated animals, comprising a composition including at least 10⁹ viable Lactobacillus bacteria per gram and at least 10⁹ viable Streptococcus faecium bacteria per gram.

69-76. (canceled)

77. A composition useful as a feed supplement for domesticated animals, comprising a composition including at least 10⁸ viable Lactobacillus bacteria per gram, and L-form Lactobacillus bacteria.

78-84. (canceled)

85. A composition useful as a feed supplement for domesticated animals, comprising a composition including at least 10⁸ viable Streptococcus faecium bacteria per gram, and L-form Lactobacillus bacteria.

86. The composition of claim 85, wherein said composition also comprises viable yeast, a nonviable yeast fermentation product, or both.

87-91. (canceled)

92. A method for enhancing milk production from a diary cow, comprising:
feeding to the dairy cow at least 10⁸ viable Lactobacillus bacteria per day and at least 10⁸ viable Streptococcus faecium bacteria per day.

93. A method for enhancing milk production from a herd of diary cows, comprising:
feeding to dairy cows in the herd at least at least 10⁸ viable Lactobacillus bacteria per day and at least 10⁸ viable Streptococcus faecium bacteria per day.

94. (canceled)

95. A composition for feeding to an animal, comprising:
viable Lactobacillus acidophilus bacteria;
viable Lactobacillus casei bacteria; and
viable Bifidobacterium bifidus bacteria.
